(19) United States
(12) Patent Application Publication
(10) Pub. No.: US 2021/0228555 A1
(43) Pub. Date: Jul. 29, 2021

(54) METHOD OF TREATMENT WITH TRADIPITANT

(71) Applicants: Vanda Pharmaceuticals Inc., Washington, DC (US); University of Kentucky Research Foundation, Lexington, KY (US)

(72) Inventors: Mihael H. Polymeropoulos, Potomac, MD (US); Gunther Birznieks, Chevy Chase, MD (US); Sharon Walsh, Lexington, KY (US)

(21) Appl. No.: 16/972,391

(22) PCT Filed: Jun. 6, 2019
(86) PCT No.: PCT/US19/35799

§ 371 Date: Dec. 4, 2020

Related U.S. Application Data
(60) Provisional application No. 62/682,831, filed on Jun. 8, 2018.

(51) Int. Cl. A61K 31/444; A61P 25/36; A61K 9/00

(52) U.S. Cl. CPC A61K 31/444; A61K 9/0053; A61P 25/36

(57) ABSTRACT

Methods of treating an individual experiencing or at risk of experiencing an undesired consequence of opioid use, and of treating an individual who is experiencing or at risk of experiencing a craving for an opioid, as well as the use of the NK-1 receptor antagonist, tradipitant, in the treatment of such an individual are disclosed herein.

### CROSS REFERENCE TO RELATED APPLICATIONS

The present application claims the benefit of U.S. provisional patent application No. 62/682,831, filed Jun. 8, 2018, which is hereby incorporated by reference in its entirety.

### BACKGROUND

The application relates generally to the use of NK-1 receptor antagonists. More particularly, the application relates the use of the NK-1 antagonist, tradipitant, for treatment of an individual experiencing or likely to experience an undesired consequence of opioid use.

Tradipitant (i.e., 2-[1-[[3,5-bis(trifluoromethyl)phenyl]methyl]-5-(4-pyridinyl)-1H-1,2,3-triazol-4-yl]-3-pyridinyl](2-chlorophenyl)-methanone or, alternatively, {2-[1-(3,5-bistrifluoromethylbenzyl)-5-pyridin-4-yl-1H-[1,2,3]triazol-4-yl]-pyridin-3-yl}-(2-chlorophenyl)-methanone) and its pharmaceutically acceptable acid addition salts (herein collectively referenced as “tradipitant”) are known as a highly potent, selective, centrally penetrating, and orally active NK-1 receptor antagonists, the free base form of which is depicted below as the compound of Formula I

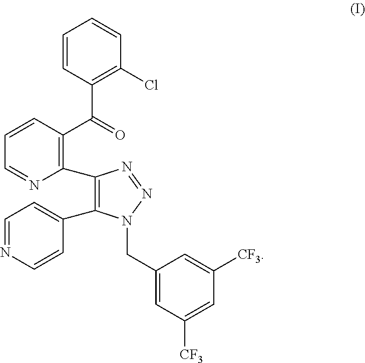 [SMILES: O=C(c1ccccc1Cl)c1cccnc1-c1nnn(Cc2cc(C(F)(F)F)cc(C(F)(F)F)c2)c1-c1ccncc1]

Crystalline Forms IV and V of the free base form of tradipitant are disclosed in U.S. Pat. No. 7,381,826.

Tradipitant, in both its free base and pharmaceutically acceptable acid addition salt is described in U.S. Pat. No. 7,320,994 as being useful in the treatment of numerous disorders related to tachykinin receptor activation including, as one of a vast array of named diseases and conditions, addiction disorders such as alcoholism Medicinal uses of tradipitant are further disclosed in international patent application publication nos. WO 2016/141341 A1 (pruritus) and WO 2019/055225 A1 (atopic dermatitis). WO 2016/141341 A1 describes the administration of tradipitant to achieve plasma concentration levels of tradipitant of 100 ng/mL or greater, 125 ng/mL or greater, 150 ng/mL or greater, 175 ng/mL or greater, 200 ng/mL or greater, and 225 ng/mL or greater for the duration of the treatment regimen, as well as administration of tradipitant at a dose of 100-400 mg/day, 100-300 mg/day, 100-200 mg/day, and 85 mg twice daily, by orally administering the tradipitant in immediate release solid dosage forms or in controlled release forms.

Opioids are known to include to both opiates (such as, e.g., heroin and morphine) and non-opiate opioids (such as, e.g., oxycodone, hydrocodone, and fentanyl) as are known in the art. Similarly, uses for opioids are known in the art, e.g., most notably as analgesics.

Opioids are known to be subject to various form of use, including both accepted forms of therapeutic use and misuse, including opioid abuse. These forms of opioid use in an individual are known to carry with them the potential to evolve over time, e.g., an individual may initiate opioid therapy for one or more accepted therapeutic uses and subsequently transition to misusing, e.g., abusing, opioids.

Misuse, including abuse, is known to result in one or more undesired aspects or consequences to an individual's psychological or physical health or wellbeing. Craving for an opioid is known to be an undesired consequence of opioid use or abuse. It can represent a primary undesired consequence of opioid use or misuse, including abuse. Craving for an opioid is known to be an undesired consequence of opioid use or abuse. It can represent a primary undesired consequence of opioid use or misuse, including abuse. Opioid abuse is also known to include untoward behavioral consequences such as the use of opioid substances prescribed for another individual, the use of opioid substances at a dose or frequency different from that prescribed, and repetitive use of opioids to produce pleasure, alleviate stress, or alter or avoid reality. Other known undesired consequences of opioid use include untoward physical, behavioral, and psychological changes, including drowsiness, mental confusion, initial euphoria followed by apathy, a sense of unease, unintentional and purposeless movement such as hand-wringing, pacing, and uncontrolled tongue movement; slowed cognition and movement, impaired judgment, nausea, constipation, depressed respiration, slurred speech, lessened pain-relieving effects over time and attendant increased pain, increased tolerance, disability, relapse, and death. Opioid misuse is known to involve the use of opioid drugs that are legally available by prescription, as well as the illegal acquisition and illegal use of unlawful substances such as heroin.

Opioids are known to produce physical dependence. Such dependence is marked by the emergence of withdrawal symptoms such as, e.g., generalized pain, muscle and bone pain, chills, cramps, dilated pupils, restlessness, anxiety, insomnia and other sleep problems, nausea, diarrhea, vomiting, cold flashes, goose bumps, uncontrollable leg movements, and severe cravings while the body adjusts to the absence or loss of the opioid substance. Individuals using or misusing including abusing opioids may or may not be addicted to opioids. Addiction to opioids is known to result in an individual's inability to control the impulse to use opioids despite the presence of undesired consequences, e.g., negative effects on personal relationships or finances.

### BRIEF DESCRIPTION OF THE INVENTION

The present invention provides tradipitant for use in the treatment of an individual, as well as a method of treating an individual who is experiencing or is at risk of experiencing an undesired consequence of opioid use, said treatment comprising administration of tradipitant to said individual, specifically administering to said individual tradipitant at a dose that is effective to achieve a plasma concentration of at least about 100 ng/mL or greater, about 125 ng/mL or greater, about 150 ng/mL or greater, about 175 ng/mL or greater, about 200 ng/mL or greater, or about 225 ng/mL during said treatment, or at a dose that is 100-400 mg/day, 100-300 mg/day, 100-200 mg/day, 150-400 mg/day, 150-300 mg/day, 150-200 mg/day, or about 170 mg/day. The dose of 170 mg/day may more particularly be 85 mg administered twice daily, e.g., administered every 12 hours.

The opioid use may be in accordance with a therapeutic regimen or may constitute a form of misuse of the opioid, as in the case of opioid abuse. The individual being treated may be theretofor opioid naïve or may be opioid-experienced, such as an individual diagnosed with opioid use disorder (OUD). As used herein, the term “individual” refers to a human being.

The undesired consequence of the opioid use may be manifest as, or otherwise include, a craving for the administration of an opioid. Such administration of tradipitant may reduce or eliminate the experience of craving or desire for the opioid in the individual being treated. Such administration of tradipitant may in other cases be prophylactic in nature and may be to an individual who is using an opioid and is therefore at risk of experiencing an undesired consequence of such use, but has not heretofore experienced such an undesired consequence.

Tradipitant for use in the treatment regimen specified above can be accomplished through formulation in an immediate release solid dosage form or a controlled release solid dosage form, such formulations being prepared using conventional methods for the preparation of pharmaceutical compositions for oral administration. Specifically, the free base form of tradipitant may be formulated using a crystalline form thereof such as Form IV or Form V as known in the art.

These and other aspects, advantages, and salient features of the invention will become apparent from the following detailed description, which, when taken in conjunction with the figures, disclose various embodiments of the invention.

### BRIEF DESCRIPTION OF THE DRAWINGS

FIGS. 1-4 illustrate the results of the study described herein with respect to tradipitant treatment effect on subjective outcomes of oxycodone administration in graphical form.

### DETAILED DESCRIPTION OF THE INVENTION

Various embodiments of the invention described herein include methods for treating an individual who is experiencing or is at risk of experiencing an undesired consequence of opioid use by administering tradipitant to the individual, tradipitant for use in the treatment of an individual who is experiencing an undesired consequence of opioid use, and a method of reducing desire (i.e. cravings) for opioids in an individual by administering tradipitant.

A method of treating an individual experiencing an undesired consequence of opioid use may first include identifying the individual to be treated. Determination of an individual for whom treatment with tradipitant is indicated can be done by a healthcare professional who is trained to identify an individual experiencing an undesired consequence of opioid abuse. In addition, individuals may self-assess their need for an intervention to address such consequences.

In some embodiments, the individual may have been opioid-naïve prior to the instance of opioid use with which the undesired consequence is associated, i.e., the individual may not have a history of having been prescribed, been administered, or self-administered opioids on a daily or regular basis. Such individuals may include, for example, individuals who are prescribed opioid analgesics by a medical provider and are using such opioid analgesics for the first time and/or according to a prescribed treatment regimen, or individuals who have recently begun self-administering opioids for any reason. In other embodiments, the individual may be opioid-experienced, i.e., may have a history of use of opioids. In further embodiments, the individual may be highly opioid experienced, and may have a diagnosis of opioid use disorder (OUD).

The method disclosed herein may include the administration of tradipitant to an individual experiencing an undesired consequence of opioid use that is consistent with characterizations of use or misuse, including abuse. Tradipitant may be administered to the individual at a dose that is effective to achieve a plasma concentration of tradipitant that is at least about 100 ng/mL or greater, about 125 ng/mL or greater, about 150 ng/mL or greater, about 175 ng/mL or greater, about 200 ng/mL or greater, or about 225 ng/mL during said treatment.

As used herein, the modifier “about” used in connection with a quantity (e.g., “about 175 ng/mL or greater”) is inclusive of the stated value and has the meaning dictated by the context (e.g., includes the degree of error associated with measurement of the particular quantity). The reference to tradipitant concentration herein is a reference to the concentration of the free base form of tradipitant.

In further embodiments, the tradipitant may be administered at a dose that is effective to both achieve and maintain the plasma concentration of tradipitant that is at least about 100 ng/mL or greater, about 125 ng/mL or greater, about 150 ng/mL or greater, about 175 ng/mL or greater, about 200 ng/mL or greater, or about 225 ng/mL during said treatment.

Plasma concentration levels disclosed herein may be achieved or achieved and maintained by orally administering tradipitant, e.g., crystalline Form IV or Form V (or a pharmaceutically acceptable acid addition salt thereof) in immediate release solid dosage forms once per day at a higher dose, in immediate release forms at a lower dose with improved bioavailability, in controlled release forms, or by orally administering the tradipitant multiple times per day, e.g., twice or more times per day, at a lower dose in immediate release or controlled release forms.

Pharmaceutically acceptable acid addition salts of tradipitant include those formed with a wide variety of organic and inorganic acids and include the physiologically acceptable salts which are often used in pharmaceutical chemistry. Such salts include the pharmaceutically acceptable salts listed in Journal of Pharmaceutical Science, 66, 2-19 (1977), which are known to the skilled artisan. Typical inorganic acids used to form such salts include hydrochloric, hydrobromic, hydroiodic, nitric, sulfuric, phosphoric, hypophosphoric, metaphosphoric, pyrophosphoric, and the like. Salts derived from organic acids, such as aliphatic mono and dicarboxylic acids, phenyl substituted alkanoic acids, hydroxyalkanoic and hydroxyalkandioic acids, aromatic acids, aliphatic and aromatic sulfonic acids, may also be used.

In embodiments disclosed herein, tradipitant may be administered to the individual at an effective dose or effective amount which, as used herein, refers to a dose or an amount of tradipitant that is effective in treating the disorders, symptoms, or consequences of opioid use described herein. In various embodiments, the effective dose of tradipitant may be, e.g., 100-400 mg/day, 100-300 mg/day, 100-200 mg/day, 150-400 mg/day, 150-300 mg/day, 150-200 mg/day, or about 170 mg/day. The reference to milligram quantities of tradipitant herein are references to its free base form. The foregoing ranges are inclusive and independently combinable, such that ranges of e.g., “100-400 mg/day” are inclusive of the endpoints thereof. The disclosed ranges are further combinable to include intermediate ranges such as, e.g., 100-150 mg/day, 150-170 mg/day, 170-200 mg/day, etc. Further, in embodiments in which tradipitant is administered at a dose of about 170 mg/day, it may more particularly be administered at a dose of 85 mg twice daily (bid), or more particularly 85 mg every 12 hours (Q12H). With regard to dosing, “bid” or twice-daily dosing typically means dosing once in the morning and once in the evening, generally no less than about 8 hours or more than about 16 hours apart, e.g., 10 to 14 hours apart, or 12 hours apart (“Q12H”).

It will be appreciated that effective plasma concentrations may also be achieved using different doses and/or different formulations, including but not limited to controlled release formulations. Regardless of the particular formulation, and whether the tradipitant is administered to the individual at a dose defined by the amount of tradipitant or by the plasma concentration level produced and/or maintained by the dose, the tradipitant may produce a significant reduction experience of an undesired consequence of opioid use such as, e.g., desire or craving for opioids. This in turn provides a useful tool for treating opioid misuse, including opioid abuse and/or addiction.

It is also recognized that one skilled in the art may affect the individual's experience of an undesired consequence of opioid use by treating an individual presently afflicted with such a consequence or by prophylactically treating an individual who is using an opioid, and has not yet experienced an undesired consequence, with an effective amount of tradipitant. Such an individual for whom prophylactic treatment may be indicated, may be said to be at risk of experiencing an undesired consequence of opioid use including opioid misuse, e.g., abuse. Since an individual continuing to use an opioid is at risk for, and may experience, an undesired consequence during such continued use, prophylactic treatment can be indicated for any such individual who is continuing with opioid use, including opioid misuse.

Thus, with respect to the present use, the terms “treatment” and “treating” are intended to encompass any process wherein there may be a slowing interrupting arresting, controlling, or stopping of the undesired consequence of opioid use. Thus, while the term includes prophylactic treatment for undesired consequences of opioid use, it does not necessarily indicate a total prophylaxis of or complete elimination of the undesired consequence.

The skilled artisan will appreciate that additional embodiments may be selected by combining the embodiments above, or by reference to the example that follows.

### Example

A double-blind, inpatient, approximately 6 week study employing a within-subject crossover design examines the effects of maintenance with tradipitant as compared to placebo on response to oxycodone. Study subjects include otherwise healthy adults reporting regular illicit opioid misuse, and history of intranasal opioid use without physical dependence. Participant characteristics are provided in Table 1 below.

TABLE 1
Participant Characteristics

| Participants (n = 8) | 8 males | |
|---|---|---|
| Age (mean/range) | 35 | (27-47) |
| Past 30-day opioid use (mean/range) | 8 | (4-16) |
| Past 30-day heroin use (mean) | 3.9 | |
| Past 30-day prescription opioids (mean) | 3.9 | |
| Age at first opioid use (mean/range) | 21 | (14-42) |
| # Years of opioid use (mean/range) | 14.3 | (5-19) |

Subjects are administered placebo or tradipitant (85 mg p.o., bid) on study days 3-17 with a washout period on days 18-23, followed by administration of placebo or tradipitant (85 mg p.o., bid) on study days 24-39. In the study, tradipitant/placebo administration is counterbalanced across subjects. On study days 3, 18, 24, and 39, subjects participate in challenge sessions (day 1 and steady state) in which they receive 0, 5, 10, or 20 mg intranasal (IN) oxycodone at 1 hr. intervals, after which effect of tradipitant on analgesia in humans is evaluated. On study days 2, 8, 11, 15, 29, 32, and 36, subjects participate in sample sessions in which subjects are administered 0, 15, or 30 mg IN oxycodone, after which effect of tradipitant on subject-rated outcomes is evaluated. Such subject-rated outcomes include responses to questions including “how much do you like the drug” “does the drug have any good effects,” “do you feel any drug effect,” and “how much do you desire opiates right now?” Further, on study days 9, 12, 16, 30, 33, and 37, subjects participate in choice sessions, in which effect of tradipitant on oxycodone self-administration is evaluated.

Results

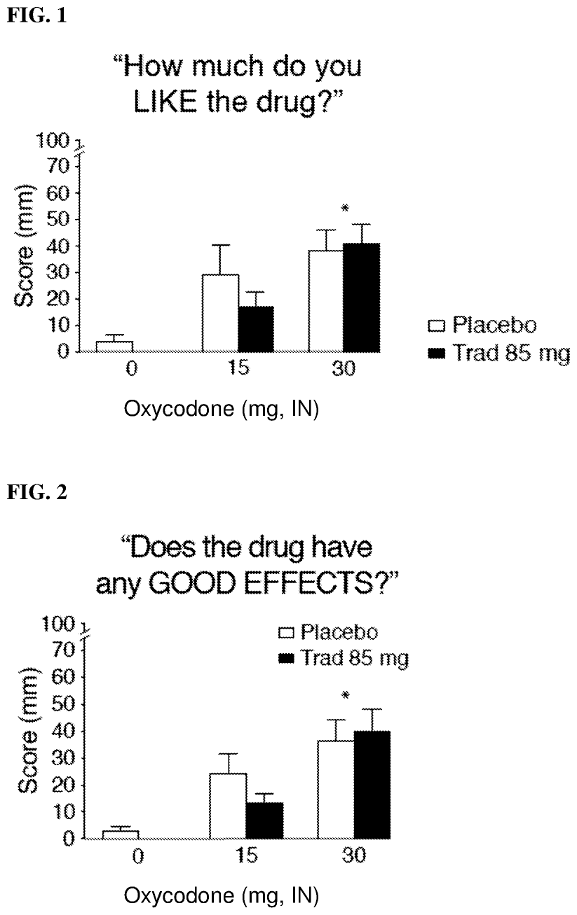

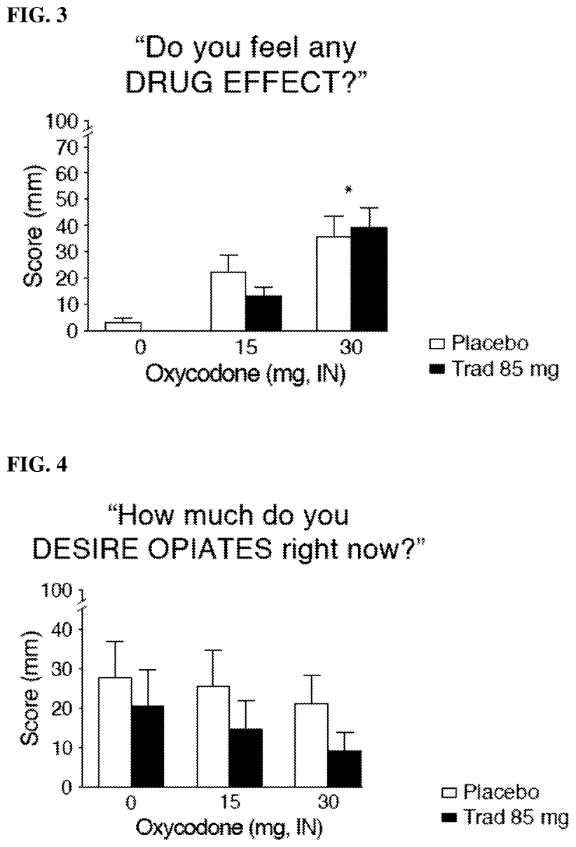

FIGS. 1-4 illustrate the results with respect to subjective outcomes in graphical form. Oxycodone produces significant and dose-dependent increases in peak ratings of liking for the drug good effects and overall drug effect (p>0.001; *indicates significant difference from 0 mg). While no significant effect of oxycodone dose on trough scores for desire for opioids is observed, there is a significant effect of tradipitant condition (p≤0.05), whereby desire, i.e. cravings, are diminished during tradipitant maintenance when compared to placebo maintenance (FIG. 4). FIGS. 1-3 further illustrate that tradipitant decreases perceptions of how much the opioid drug was liked, how good the effects of the drug were, and how much of a drug effect was felt at some opioid drug doses, e.g., at 15 mg IN oxycodone.

Accordingly, tradipitant maintenance is found to decrease opioid drug craving, and at some doses decreases pleasurable and drug seeking sensations. In particular, tradipitant maintenance significantly (p<0.05) reduces desire for opioid drugs compared to placebo.

1. A method of treating an individual who is experiencing or is at risk of experiencing an undesired consequence of opioid use, comprising:
administering to said individual tradipitant:
at a dose that is effective to achieve a plasma concentration of at least about 100 ng/mL or greater during said treatment, or
at a dose that is 150-400 mg/day.

2. (canceled)

3. The method of claim 1, wherein the plasma concentration level is about 125 ng/mL or greater.

4. The method of claim 3, wherein the plasma concentration level is about 150 ng/mL or greater.

5. The method of claim 4, wherein the plasma concentration level is about 175 ng/mL or greater.

6. The method of claim 5, wherein the plasma concentration level is about 200 ng/mL or greater.

7. The method of claim 6, wherein the plasma concentration level is about 225 ng/mL or greater.

8-11. (canceled)

12. The method of claim 1, wherein the dose is 150-300 mg/day.

13. The method of claim 12, wherein the dose is 150-200 mg/day.

14. The method of claim 13, wherein the dose is about 170 mg/day.

15. The method of claim 14, wherein the dose is 85 mg twice daily (bid).

16. (canceled)

17. The method of claim 1, wherein the opioid use further comprises opioid misuse.

18. The method of claim 1, wherein the individual is opioid-experienced.

19. The method of claim 1, wherein the individual is diagnosed with opioid use disorder (OUD).

20. The method of claim 1, wherein the undesired consequence of opioid use or opioid abuse is craving for administration of an opioid.

21. The method of claim 1, wherein the individual being treated is experiencing an undesired consequence of opioid use.

22. The method of claim 1, wherein the individual being treated is at risk of experiencing an undesired consequence of opioid use.

23-42. (canceled)

43. The method of claim 1, wherein the tradipitant is in an immediate release solid dosage form or a controlled release solid dosage form, and wherein the tradipitant is in crystalline Form IV or Form V.

44-47. (canceled)

48. A method of treating an individual who is experiencing or is at risk of experiencing a craving for an opioid, comprising:
administering to said individual tradipitant:
at a dose that is effective to achieve a plasma concentration of at least about 100 ng/mL or greater during said treatment, or
at a dose that is 150-400 mg/day.

49. The method of claim 48, wherein the individual being treated is experiencing an undesired consequence of opioid use.

50. The method of claim 48, wherein the individual being treated is at risk of experiencing an undesired consequence of opioid use.
